(19) United States
(12) Patent Application Publication
(10) Pub. No.: US 2011/0072524 A1
(43) Pub. Date: Mar. 24, 2011

(54) Transgenic Mouse Lacking Endogenous FVIII and VWF - A Model of Hemophilia A

(75) Inventors: Peter Turecek, Klosterneuburg (AT); Raffi Udin Ahmad, Vienna (AT); Herbert Gritsch, Vienna (AT); Eva-Maria Muchitsch, Vienna (AT); Hans-Peter Schwarz, Vienna (AT)

(73) Assignees: BAXTER INTERNATIONAL INC., Deerfield, IL (US); BAXTER HEALTHCARE S.A., Glattpark (Opfikon) (CH)

(21) Appl. No.: 12/850,243

(22) Filed: Aug. 4, 2010

Related U.S. Application Data
(60) Provisional application No. 61/231,230, filed on Aug. 4, 2009.

(51) Int. Cl. A61K 38/36; A01K 67/00; A01K 67/027; A61K 49/00; A61P 7/04

(52) U.S. Cl. 800/3; 800/13; 800/14; 800/18; 800/9; 514/14.1

(57) ABSTRACT

The present invention relates, generally, to a transgenic non-human animal model of hemophilia A, wherein the transgenic animal is deficient in endogenous Factor VIII and endogenous von Willebrand Factor, and methods to treat hereditary or acquired hemophilia A or von Willebrand Disease (VWD) by administration of exogenous human VWF.

### CROSS-REFERENCE TO RELATED APPLICATIONS

The present application claims the priority benefit of U.S. Provisional Patent Application No. 61/231,230, filed Aug. 4, 2009, hereby incorporated by reference.

### FIELD OF THE INVENTION

The present invention relates generally to transgenic non-human animals lacking Factor VIII (FVIII) and von Willebrand Factor (VWF) useful for the study of blood coagulation disorders, such as hemophilia A.

### BACKGROUND OF THE INVENTION

Blood coagulation is a complex process including the sequential interaction of a series of components, in particular of fibrinogen, Factor II, Factor V, Factor VII, Factor VIII, Factor IX, Factor X, Factor XI, Factor XII and von Willebrand's factor. The loss of one of these components or the inhibition of its functionality may cause either an increased tendency of blood coagulation or an inability to clot, which may be life-threatening in some patients.

Factor VIII is a protein found in blood plasma which acts as a cofactor in the cascade of reactions leading to blood coagulation. A deficiency in the amount of Factor VIII activity in the blood results in the clotting disorder hemophilia A, an inherited condition primarily affecting males. Hemophilia A is currently treated with therapeutic preparations of Factor VIII derived from human plasma or manufactured using recombinant DNA technology. Such preparations are administered either in response to a bleeding episode or at frequent, regular intervals to prevent uncontrolled bleeding (prophylaxis).

Von Willebrand factor (VWF) circulates in plasma complexed with Factor VIII. VWF complexed with Factor VIII stabilizes the Factor VIII protein and protects it from proteolytic degradation. Due to its function in platelet aggregation, VWF also directly interferes in blood coagulation. Von Willebrand deficiency (VWD) (also known as von Willebrand syndrome) results from either a deficiency or overexpression of VWF. Deficiency of VWF results in a disease similar to hemophilia due to the rapid degradation of Factor VIII lacking VWF cofactor.

Conventional methods for a therapy of hemophilia A and von Willebrand syndrome use Factor VIII or VWF recovered from plasma or produced by recombinant sources, and there are a number of attempts to treat patients with purified Factor VIII, VWF or Factor VIII/VWF-complex.

Transgenic animal technology presents a unique opportunity to study the characteristics of human proteins in non-human animals. Recombinant DNA and genetic engineering techniques have made it possible to interrupt expression of an endogenous gene, or to introduce and express a desired sequence or gene in a recipient animal making it possible to study the effects of a particular molecule in vivo and study agents that bind to the molecule. One method for producing transgenic mice requires the recovery of fertilized eggs from newly mated female mice and microinjection of the DNA of the gene of interest for expression in the animal or to interfere with endogenous gene expression into the male pronucleus of the egg. The microinjected eggs are then implanted in the oviducts of one-day pseudopregnant foster mothers and allowed to proceed to term. See, for example, Wagner et al. Proc Natl. Acad. Sci. U.S.A. 78:6376-6380 (1981), U.S. Pat. Nos. 4,873,191, and 7,294,755. Another procedure uses embryonic stem cells that are transfected with the gene of interest. Transfected embryonic stem cells are then injected into mouse blastocysts where they take part in the formation of all tissues, including the germ line, thus generating transgenic offspring. This approach, in combination with the homologous recombination technology, offers the possibility of altering embryonic stem cells in a controlled manner and therefore, of generating transgenic mice with a predetermined genome. See, for example: Baribault and Kemler. Embryonic stem cell culture and gene targeting in transgenic mice. Mol Biol Med. 6:481-92, 1989; Ledermann B. Embryonic stem cells and gene targeting. Exp Physiol. 85:603-13, 2000; Moreadith and Radford. Gene targeting in embryonic stem cells: the new physiology and metabolism. J Mol. Med. 75:208-16, 1997.

Transgenic mice may be generated to express or overexpress a protein of interest (knock-in mice) or may be generated to delete a gene of interest (knock-out mice). Transgenic mice which express a human protein molecule allow for study of the human molecules in vivo. For example, Shi et al. (J Clin Invest. 116:1974-82, 2006) describe transgenic mice expressing a modified human FVIII protein (lacking the B-domain) designed to circumvent the problem of FVIII inhibitory antibodies which inhibit the activity of recombinant FVIII. VWF knockout mice exhibiting symptoms similar to human VWD are disclosed in Pergolizzi et al., (Blood. 108:862-9, 2006), which describes administration of a gene therapy vector comprising murine VWF to correct VWF deficiency.

Thus, there exists a need in the art to develop improved methods to study the activity of human blood coagulation factors in vivo without study on human patients. Further, there remains a need in the art to determine the therapeutic effects of administration of exogenous therapeutic protein to a patient having a blood coagulation disorder such as hemophilia A.

### SUMMARY OF THE INVENTION

The present invention addresses one or more needs in the art relating to treatment of blood clotting disorders by providing a transgenic non-human animal useful to study the effects of therapeutic regimens in vivo, as well as development of methods to treat human blood clotting disorders using administration of exogenous human blood clotting factors.

In one embodiment, the invention provides a transgenic non-human animal having a genome lacking a functional, endogenous Factor VIII (FVIII) gene and lacking a functional, endogenous von Willebrand Factor (VWF) gene. In one aspect, the transgenic animal has a genome comprising a human transgene polynucleotide sequence encoding human VWF. Optionally, the polynucleotide sequence is operably linked to a promoter polynucleotide sequence and/or the polynucleotide sequence comprises a polyadenylation polynucleotide sequence. In various aspects, the animal is a rodent and in one specific aspect, the animal is a mouse.

The invention also provides a non-human transgenic animal having a genome lacking a functional, endogenous Factor VIII (FVIII) gene and lacking a functional, endogenous von Willebrand Factor (VWF) gene, the genome comprising a polynucleotide encoding a human von Willebrand Factor, the human VWF having physiological activity of the human VWF, the transgenic mammal having in its genome an exogenous transgene construct comprising transcriptional regulatory polynucleotide sequences, DNA encoding said human VWF, and a polyadenylation signal, wherein the transcriptional regulatory polynucleotide sequences, the DNA encoding said human VWF, and the polyadenylation signal are operably linked in the exogenous gene construct to obtain production of the human VWF or fragment thereof in the transgenic animal. In one aspect, the transcriptional regulatory polynucleotide sequences are selected from the group consisting of 5′ transcriptional regulatory polynucleotide sequences, 3′ transcriptional regulatory polynucleotide sequences, internal transcriptional regulatory polynucleotide sequences, and combinations thereof. In a specific aspect, the 5′ regulatory sequence is a promoter, optionally comprising an enhancer region.

In various embodiments of the transgenice animal provided, the animal is an experimental model of human acquired hemophilia A.

Also provided is a method for assessing the effects of exogenous human von Willebrand factor (VWF) on a mammal deficient in endogenous Factor VIII and endogenous VWF comprising administering to an animal as described hereinabove a therapeutically effective amount of human VWF and measuring a clinical readout of VWF activity, wherein an improvement in clinical readout indicates a therapeutic effect of exogenous VWF. In certain aspects of the method, the VWF is administered intraperitoneally or subcutaneously. In other aspects, the VWF is administered in a range from 50 VWF:Ag U/kg to 500 U/kg. In still other aspects, the clinical readout of VWF activity is selected from the group consisting of blood composition, platelet aggregation, platelet adhesion and platelet activation.

The invention further provides a method for treating a subject having hemophilia A comprising administering an exogenous multimeric human von Willebrand Factor in an amount effective to ameliorate a symptom of hemophilia A, wherein the VWF is administered intraperitoneally or subcutaneously. In one aspect, the symptom is selected from the group consisting of bleeding disorder, autoimmune disease, aberrant platelet aggregation, aberrant platelet adhesion and aberrant platelet activation. In another aspect, the VWF is administered in a range from 50 VWF:Ag U/kg to 500 U/kg.

In various embodiments of the methods provided hereinabove, the VWF is administered in combination with human Factor VIII.

### BRIEF DESCRIPTION OF THE FIGURES

FIG. 1 shows the levels of recombinant VWF antigen in plasma of FVIIIxVWD double knockout mice after administration of 200 rVWF/kg (FIG. 1A) or 400 rVWF/kg (FIG. 1B).

FIG. 2 depicts low resolution SDS-PAGE (FIG. 2A) or high resolution SDS PAGE (FIG. 2B) illustrating that intraperitoneally injected rVWF is detected as high-molecular weight multimers in the plasma of FVIIIxVWD mice.

FIG. 3 illustrates that plasma derived VWF injected intraperitoneally is detected in the circulation of FVIIIxVWD knockout mice.

FIG. 4 shows that plasma derived VWF injected intraperitoneally is preserved in the circulation as intact multimers. (A) low resolution SDS-PAGE; (B) high resolution SDS-PAGE.

FIG. 5 shows that rVWF:Ag is detectable in the plasma for up to 24 hours after intraperitoneal injection of 200 IU rFVIII/kg and 200 IU VWF:Ag/kg.

FIG. 6 shows that FVIII was detectable in the plasma for up to 9 hours after intraperitoneal injection of 200 IU rFVIII/kg and 200 IU VWF:Ag/kg, but was not detectable after 24 hours.

FIG. 7 illustrates the blood levels of FVIII (FIG. 7A) and rVWF:Ag (FIG. 7B) for up to 48 hours after intraperitoneal injection of 200 IU rFVIII/kg and 200 IU VWF:Ag/kg. FVIII and rVWF were not detectable in the circulation after 46 hours.

### DETAILED DESCRIPTION OF THE INVENTION

The present invention addresses the need in the art for improved methods and compositions useful to treat blood clotting disorders. The present invention also provides transgenic animals lacking endogenous blood clotting factors, and in some instances expressing human clotting factors in place of a blood clotting factor endogenous to the animal, in order to address the efficacy of therapeutic compositions in the treatment of human blood clotting disorders.

### Definitions

Unless otherwise defined, all technical and scientific terms used herein have the same meaning as commonly understood by one of ordinary skill in the art to which this invention belongs. The following references provide one of skill with a general definition of many of the terms used in this invention: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

It is noted here that, as used in this specification and the appended claims, the singular forms “a,” “an,” and “the” include plural reference unless the context clearly dictates otherwise.

As used herein, the following terms have the meanings ascribed to them unless specified otherwise

As used herein, a “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

As used herein a “gene” refers to a DNA sequence that encodes, or a particular sequence of amino acids which comprise, all or part of one or more polypeptides, proteins or enzymes, and may or may not include introns, and regulatory DNA sequences, such as promoter or enhancer sequences, 5′-untranslated region, or 3′-untranslated region which affect, for example, the conditions under which the gene is expressed. Some genes, which are not structural genes, may be transcribed from DNA to RNA, but are not translated into an amino acid sequence. Other genes may function as regulators of structural genes or as regulators of DNA transcription.

As used herein, a “transgene” refers to an exogenous gene introduced into the genome of another organism. A human transgene is a human gene introduced into a non-human mammal.

As used herein a “promoter” or “promoter sequence” is a DNA regulatory region capable of binding RNA polymerase in a cell and initiating transcription of a coding sequence. In one aspect, the promoter sequence is bound at its 3′ terminus by a transcription initiation site and extends upstream (5′ direction) to include the minimum number of bases or elements necessary to initiate transcription at levels detectable above background. In a related aspect, within the promoter sequence is found a transcription initiation site (conveniently defined for example, by mapping with nuclease S1), as well as protein binding domains (consensus sequences) responsible for the binding of RNA polymerase. The promoter may be operatively associated with other expression control sequences, including enhancer and repressor sequences.

In one aspect, promoters used to control gene expression include, but are not limited to, cytomegalovirus (CMV) promoter (U.S. Pat. No. 5,385,839 and No. 5,168,062), the SV40 early promoter region (Benoist and Chambon, Nature 290:304-310, 1981), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. USA 78:1441-1445, 1981), the regulatory sequences of the metallothionein gene (Brinster et al., Nature 296:39-42, 1982); promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit neuronal or brain specific expression, such as the gonadotropin releasing hormone gene control region which is active in the hypothalamus (Mason et al., Science 234:1372-1378, 1986), the Thy1.2 “pan-neuronal” promoter, and synapsin I promoter (Howland et al., Brain Neurobiol Aging 16: 685-699, 1995), active in neurons. It is also contemplated that the promoter is an endogenous blood clotting factor promoter. The worker of ordinary skill in the art will understand that any promoter known in the art is useful, and that the cell type in which expression is desired can dictate use of a particular promoter.

As used herein a coding sequence is “under the control of,” “operably linked to” or “operatively associated with” transcriptional and translational control sequences in a cell when RNA polymerase transcribes the coding sequence into RNA, which is then trans-RNA spliced (if it contains introns) and translated, in the case of mRNA, into the protein encoded by the coding sequence.

As used herein the terms “express” and “expression” refer to allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed.” An expression product is, in various aspects, characterized as intracellular, extracellular or secreted. The term “intracellular” means inside a cell. The term “extracellular” means outside a cell, such as a transmembrane protein. A substance is “secreted” by a cell if it appears in significant measure outside the cell, from somewhere on or inside the cell.

As used herein “transfection” refers to the introduction of a foreign nucleic acid into a cell. The term “transformation” refers to the introduction of a “foreign” (i.e. exogenous, heterologous, extrinsic or extracellular) gene, DNA or RNA sequence to an embryonic stem (ES) cell or pronucleus, so that the cell will express the introduced gene or sequence to produce a desired substance in a transgenic animal.

As used herein the terms “vector,” “cloning vector” and “expression vector” refer to a vehicle by which a DNA or RNA sequence (e.g., a foreign gene) is introduced into a host cell so as to transform the host and promote expression (e.g., transcription and translation) of the introduced sequence. The term “vector” is used herein interchangeably with the term “plasmid.”

As used herein “selectable marker” refers to a gene encoding an enzyme or other protein that confers upon the cell or organism in which it is expressed an identifiable phenotypic change such as resistance to a drug, antibiotic or other agent, such that expression or activity of the marker is selected for (for example, but without limitation, a positive marker, such as the neo gene) or against (for example, and without limitation, a negative marker, such as the dipteheria gene). A heterologous selectable marker refers to a selectable marker gene that has been inserted into the genome of an animal in which it would not normally be found.

Examples of selectable markers include, but are not limited to, an antibiotic resistance gene such as neomycin (neo), puromycin (Puro), diphtheria toxin, phcsphotransferase, hygromycin phosphotransferase, xanthineguanine phosphoribosyl transferase, the Herpes simplex virus type 1 thymidine kinase, adenine phosphoribosyltransferase and hypoxanthine phosphonbosyltransferase. The worker of ordinary skill in the art will understand any selectable marker known in the art is useful in the method.

As used herein “heterologous” refers to a combination of elements not naturally occurring in itself and/or found in a non-natural environment. For example, heterologous DNA refers to DNA not naturally located in the cell, or in a chromosomal site of the cell. It is contemplated that the heterologous DNA includes a gene foreign to the cell. A heterologous expression regulatory element is such an element operatively associated with a different gene than the one it is operatively associated with in nature.

As used herein, the term “homologous” refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell 50:667, 1987). Such proteins (and their encoding genes) have sequence homology, as reflected by their sequence similarity, whether in terms of percent similarity or the presence of specific residues or motifs at conserved positions.

Optimal alignment of sequences for comparison is conducted, for example and without limitation, by the local homology algorithm of Smith & Waterman, Adv. Appl. Math. 2:482, 1981; by the homology alignment algorithm of Needleman & Wunsch, J. Mol. Biol. 48:443, 1970; by the search for similarity method of Pearson & Lipman, Proc. Natl. Acad. Sci. USA 85:2444, 1988; by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra). Another example of algorithm that is suitable for determining percent sequence identity and sequence similarity is the BLAST algorithm, which is described in Altschul et al., J. Mol. Biol. 215:403-410, 1990. Software for performing BLAST analyses is publicly available through the National Center for Biotechnology Information. In addition to calculating percent sequence identity, the BLAST algorithm also performs a statistical analysis of the similarity between two sequences (see, e.g., Karlin & Altschul, Proc. Nat'l. Acad. Sci. USA 90:5873-5787, 1993).

As used herein “endogenous” refers to a polypeptide or polynucleotide or other compound that is expressed naturally in the host organism, or originates within a cell, tissue or organism. “Exogenous” refers to a polypeptide, polynucleotide or other compound that originates outside a cell, tissue or organism.

As used herein a “polypeptide” refers to a polymer composed of amino acid residue linked via peptide bonds. Synthetic polypeptides are synthesized, in one aspect, using an automated polypeptide synthesizer. The term “protein” typically refers to large polypeptides. The term “peptide” typically refers to short polypeptides.

As used herein a “fragment” of a polypeptide refers to any portion of the polypeptide smaller than the full-length polypeptide or protein expression product. Fragments are typically deletion analogs of the full-length polypeptide wherein one or more amino acid residues have been removed from the amino terminus and/or the carboxy terminus of the full-length polypeptide. Accordingly, “fragments” are a subset of deletion analogs described below.

As used herein an “analog” refers to a polypeptide substantially similar in structure and having the same biological activity, albeit in certain instances to a differing degree, to a naturally-occurring molecule. Analogs differ in the composition of their amino acid sequences compared to the naturally-occurring polypeptide from which the analog is derived, based on one or more mutations involving (i) deletion of one or more amino acid residues at one or more termini of the polypeptide and/or one or more internal regions of the naturally-occurring polypeptide sequence, (ii) insertion or addition of one or more amino acids at one or more termini (typically an “addition” analog) of the polypeptide and/or one or more internal regions (typically an “insertion” analog) of the naturally-occurring polypeptide sequence or (iii) substitution of one or more amino acids for other amino acids in the naturally-occurring polypeptide sequence. Substitutions are conservative or non-conservative based on the physico-chemical or functional relatedness of the amino acid that is being replaced and the amino acid replacing it. As noted herein, deletion analogs wherein one or more amino acid residues at one or both termini of the naturally occurring amino acid sequence are removed include fragments. Also, addition analogs, wherein all or part of another protein or polypeptide sequence are added to one or both termini of the naturally occurring amino acid sequence, include fusion proteins.

As used herein an “allelic variant” refers to any of two or more polymorphic forms of a gene occupying the same genetic locus. Allelic variations arise naturally through mutation, and may result in phenotypic polymorphism within populations. In certain aspects, gene mutations are silent (no change in the encoded polypeptide) or may encode polypeptides having altered amino acid sequences. “Allelic variants” also refer to cDNAs derived from mRNA transcripts of genetic allelic variants, as well as the proteins encoded by them.

As used herein a “variant” refers to a polypeptide, protein or analog thereof that is modified to comprise additional chemical moieties not normally a part of the molecule. Such moieties, in various aspects, modulate the molecule's solubility, absorption, and/or biological half-life. The moieties in various other aspects, alternatively decrease the toxicity of the molecule and eliminate or attenuate any undesirable side effect of the molecule, etc. Moieties capable of mediating such effects are disclosed in Remington's Pharmaceutical Sciences (1980). Procedure for coupling such moieties to a molecule are well known in the art. For example, the variant may be a blood clotting factor having a chemical modification which confers a longer half-life in vivo to the protein. In one embodiment, the polypeptides are modified by addition of a water soluble polymer known in the art. In a related embodiment, polypeptides are modified by glycosylation, PEGylation, and/or polysialylation.

As used herein a “detectable moiety” or “label” refers to a composition detectable by spectroscopic, photochemical, biochemical, immunochemical, chemical or other means. For example, useful labels include ³²P, ³⁵S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that is used to quantitate the amount of bound detectable moiety in a sample.

### Blood Clotting Factors

Factor VIII (FVIII) is a blood plasma glycoprotein of about 260 kDa molecular mass produced in the liver of mammals. It is acritical component of the cascade of coagulation reactions that lead to blood clotting. Within this cascade is a step in which Factor IXa, in conjunction with FVIII, converts Factor X to an activated form, Factor Xa. FVIII acts as a cofactor at this step, being required with calcium ions and phospholipid for the activity of Factor IXa. The two most common hemophilic disorders are caused by a deficiency of functional FVIII (hemophilia A, about 80% of all cases) or functional Factor IXa (hemophilia B or Christmas Factor disease).

Until recently, the standard treatment of hemophilia A involved frequent infusion of preparations of FVIII concentrates derived from the plasmas of human donors. While this replacement therapy is generally effective, such treatment puts patients at risk for virus-transmissible diseases such as hepatitis and AIDS. Although this risk has been reduced by further purification of FVIII from plasma by immunopurification using monoclonal antibodies, and by inactivating viruses by treatment with either an organic solvent or heat, such preparations have greatly increased the cost of treatment and are not without risk. For these reasons, patients have been treated episodically, rather than prophylactically.

An important advance in the treatment of hemophilia A has been the isolation of cDNA clones encoding the complete 2,351 amino acid sequence of human FVIII (see, Wood et al, Nature, 312: 330 (1984) and U.S. Pat. No. 4,757,006, Jul. 12, 1988) and the provision of the human FVIII gene DNA sequence and recombinant methods for its production. However, patients receiving recombinant FVIII may still develop FVIII-specific antibodies which interfere with treatment of the disease. Factor VIII products for the treatment of hemophilia include, but are not limited to: ADVATE® (Antihemophilic Factor (Recombinant), Plasma/Albumin-Free Method, rAHF-PFM), recombinant Antihemophilic Factor (BIOCLATE™, GENARC®, HELIXATE FS®, KOATE®, KOGENATE FS®, RECOMBINATE®): MONOCLATE-P®, purified preparation of Factor VIII:C, Antihemophilic Factor/von Willebrand Factor Complex (Human) HUMATE-P® and ALPHANATE®, Anti-hemophilic Factor/von Willebrand Factor Complex (Human); and HYATE C®, purified pig Factor VIII.

von Willebrand Factor exists in plasma in a series of multimer forms of a molecular weight of from 1×10⁶ to 20×10⁶ Dalton. VWF is a glycoprotein primarily formed in the endothelial cells of mammals and subsequently secreted into circulation. In this connection, starting from a polypeptide chain having a molecular weight of approximately 220 kD, a VWF dimer having a molecular weight of 550 kD is produced in the cells by the formation of several sulfur bonds. Further polymers of the VWF with increasing molecular weights, up to 20 million Dalton, are formed from the VWF dimers by linking. It is presumed that particularly the high-molecular VWF multimers have an essential importance in blood coagulation.

VWF syndrome manifests clinically when there is either an underproduction or an overproduction of VWF or an autoimmune disease with inhibitory antibodies to VWF (acquired VWD). Overproduction of VWF causes increased thrombosis (formation of a clot or thrombus inside a blood vessel, obstructing the flow of blood) while reduced levels of, or lack of, high-molecular forms of VWF causes increased bleeding and an increased bleeding time due to inhibition of platelet aggregation and wound closure.

A VWF deficiency may also cause a phenotypic hemophilia A since VWF is an essential component of functional Factor VIII. In these instances, the half-life of Factor VIII is reduced to such an extent that its function in the blood coagulation cascade is impaired. Patients suffering from von Willebrand disease (VWD) or VWF syndrome frequently exhibit a Factor VIII deficiency. In these patients, the reduced Factor VIII activity is not the consequence of a defect of the X chromosomal gene, but an indirect consequence of the quantitative and qualitative change of VWF in plasma. The differentiation between hemophilia A and VWD may normally be affected by measuring the VWF antigen or by determining the ristocetin-cofactor activity. Both the VWF antigen content and the ristocetin cofactor activity are lowered in most VWD patients, whereas they are normal in hemophilia A patients. VWF products for the treatment of VWF syndrome include, but are not limited to: HUMATE-P; and, IMMUNATE®, ALPHANATE®, INNOBRAND®, and 8Y®, which therapies comprising FVIII/VWF concentrate from plasma.

Additional blood factors amendable to use in methods of the invention include without limitation Factor II (also known in the art as thrombin) (Genbank Accession No. NP_{—}000497), deficiencies of which cause thrombosis and dysprothrombinemia; Factor V, (Genbank Accession No. NP_{—}000121), deficiencies of which cause hemorrhagic diathesis or a form of thrombophilia, which is known as activated protein C resistance, Factor XI (Genbank Accession No. NP_{—}000119), deficiencies of which cause Rosenthal's syndrome (hemophilia C), and Factor XIII subunit A (Genbank Accession No. NP_{—}000120) and subunit B (Genbank Accession No. NP_{—}001985), deficiencies of which are characterized as a type I deficiency (deficiency in both the A and B subunits) and type II deficiency (deficiency in the A subunit alone), either of which can result in a lifelong bleeding tendency, defective wound healing, and habitual abortion Factor XII (Genbank Accession No. NP_{—}000496); protein C (Genbank Accession No. NP_{—}000303); antithrombin III (Genbank Accession No. NP_{—}000479), and activated forms thereof.

### Fragments, Variants and Analogs of Human Blood Clotting Factors

In order to assess the therapeutic efficacy of a human blood clotting factor protein in the treatment of a blood clotting disorder, the human blood clotting factor polypeptide or a fragment, variant or analog thereof, is administered to a transgenic mouse described herein.

Methods for preparing polypeptide fragments, variants or analogs are well-known in the art. Fragments of a polypeptide are prepared using, without limitation, including enzymatic cleavage (e.g., trypsin, chymotrypsin) and also using recombinant means to generate a polypeptide fragment having a specific amino acid sequence. Using a polynucleotide encoding a desired fragment, polypeptide fragments may be generated comprising a region of the protein having a particular activity, such as a ligand binding domain, a receptor binding domain, a dimerization or multimerization domain, or any other identifiable domain known in the art.

Methods of making polypeptide analogs are also well-known. Amino acid sequence analogs of a polypeptide are substitutional, insertional or deletion variants. Deletion analogs, including fragments of a polypeptide, lack one or more residues of the native protein which are not essential for function or immunogenic activity. Insertional analogs involve the addition of material at a non-terminal point in the polypeptide. This may include the insertion of an immunoreactive epitope or simply a single residue.

Analogs may be substantially homologous or substantially identical to the blood clotting factors from which they are derived and described herein. Contemplated analogs are those which retain at least some of the biological activity of the wild-type polypeptide, e.g. blood clotting activity.

Substitution analogs typically exchange one amino acid of the wild-type for another at one or more sites within the protein, and may be designed to modulate one or more properties of the polypeptide, as described herein, without the loss of other functions or properties. In one aspect, substitutions are conservative substitutions. By “conservative amino acid substitution” is meant substitution of an amino acid with an amino acid having a side chain of a similar chemical character. Similar amino acids for making conservative substitutions include those having an acidic side chain (glutamic acid, aspartic acid); abasic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine).

Polynucleotides encoding fragments and analogs may be readily generated by a worker of skill to encode biologically active fragments, variants, or analogs of the naturally-occurring molecule that possess the same or similar biological activity to the naturally-occurring molecule. This may be done by PCR techniques, digestion of DNA encoding molecule, and the like. Thus, one of skill in the art will be able to generate single base changes in the DNA strand to result in an altered codon and a missense mutation.

Exemplary blood clotting factor analogs are described, for example, in U.S. Pat. No. 6,346,513, U.S. Pat. No. 6,316,226, U.S. Pat. No. 6,156,888, U.S. Pat. No. 6,130,203, and U.S. Pat. No. 6,958,322, herein incorporated by reference.

Human blood clotting factor variants contemplated include polypeptides chemically modified by such techniques as ubiquitination, glycosylation, conjugation to therapeutic or diagnostic agents, labeling (e.g., with radionuclides or various enzymes), covalent polymer attachment such as PEGylation (derivatization with polyethylene glycol), introduction of non-hydrolyzable bonds, and insertion or substitution by chemical synthesis of amino acids such as ornithine, which do not normally occur in human proteins. Variants retain the binding properties of non-modified molecules of the invention.

Preparing PEGylated blood clotting factor analogs will generally comprise the steps of (a) reacting the polypeptide with polyethylene glycol (such as a reactive ester or aldehyde derivative of PEG) under conditions whereby the binding construct polypeptide becomes attached to one or more PEG groups, and (b) obtaining the reaction product(s). In general, the optimal reaction conditions for the acylation reactions will be determined based on known parameters and the desired result. For example, the larger the ratio of PEG: protein, the greater the percentage of poly-PEGylated product. In some embodiments, the binding construct will have a single PEG moiety at the N-terminus.

Polyethylene glycol (PEG) may be attached to the blood clotting factor to provide a longer half-life in vivo. The PEG group may be of any convenient molecular weight and may be linear or branched. The average molecular weight of the PEG ranges from about 2 kiloDalton (“kD”) to about 100 kDa, from about 5 kDa to about 50 kDa, or from about 5 kDa to about 10 kDa. The PEG groups are attached to the blood clotting factor via acylation or reductive alkylation through a natural or engineered reactive group on the PEG moiety (e.g., an aldehyde, amino, thiol, or ester group) to a reactive group on the blood clotting factor (e.g., an aldehyde, amino, or ester group) or by any other technique known in the art.

Polypeptide variants useful in the methods of the present invention include polypeptide comprising polysialylate (PSA) moieties. Methods for preparing polysialylated polypeptide are described in United States Patent Publication 20060160948 and Saenko et al., Haemophilia 12:42-51, 2006.

It is further contemplated that the human blood clotting factor for use in the methods of the invention include a fusion protein with a second agent which is a polypeptide. In one embodiment, the second agent which is a polypeptide, without limitation, is an enzyme, a growth factor, a cytokine, a chemokine, a cell-surface receptor, the extracellular domain of a cell surface receptor, a cell adhesion molecule, or fragment or active domain of a protein described above. In a related embodiment, the second agent is a blood clotting factor such as Factor VIII, Factor VII, Factor IX and von Willebrand factor. The fusion protein contemplated is made by chemical or recombinant techniques well-known in the art.

### Recombinant VWF

One form of useful rVWF has at least the property of in vivo-stabilizing, e.g., binding, of at least one Factor VIII (FVIII) molecule and having optionally a glycosylation pattern which is pharmacologically acceptable. Specific examples thereof include VWF without the A2 domain which are thereby resistant to proteolysis (Lankhof et al., Thromb. Haemost. 77: 1008-1013, 1997), and the VWF fragment from Val 449 to Asn 730 including the glycoprotein lb-binding domain and binding sites for collagen and heparin (Pietu et al., Biochem. Biophys. Res. Commun. 164: 1339-1347, 1989). The determination of the ability of a VWF to stabilize at least one FVIII molecule can be carried out in VWF-deficient mammals according to methods known in the state in the art.

The rVWF useful in the present invention may be produced by any method known in the art. One specific example is disclosed in WO86/06096 published on Oct. 23, 1986 and U.S. patent application Ser. No. 07/559,509, filed on Jul. 23, 1990, which is incorporated herein by reference with respect to the methods of producing recombinant VWF. Thus, methods are known in the art for (i) the production of recombinant DNA by genetic engineering, e.g. via reverse transcription of RNA and/or amplification of DNA, (ii) introducing recombinant DNA into prokaryotic or eukaryotic cells by transfection, e.g. via electroporation or microinjection, (iii) cultivating said transformed cells, e.g. in a continuous or batchwise manner, (iv) expressing VWF, e.g. constitutively or upon induction, and (v) isolating said VWF, e.g. from the culture medium or by harvesting the transformed cells, in order to (vi) obtain purified rVWF, e.g. via anion exchange chromatography or affinity chromatography. A recombinant VWF may be made in transformed host cells using recombinant DNA techniques well known in the art. For instance, sequences coding for the polypeptide could be excised from DNA using suitable restriction enzymes.

Alternatively, the DNA molecule is synthesized using chemical synthesis techniques, such as the phosphoramidate method. Also, a combination of these techniques could be used.

### Blood Clotting Factor Compositions for Administration

To administer blood clotting factor polypeptides (including fragments, analogs or variants) described herein to test subjects, blood clotting factor polypeptides are formulated in a composition comprising one or more pharmaceutically acceptable carriers. The phrase “pharmaceutically or pharmacologically acceptable” refers to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like.

In addition, compounds may form solvates with water or common organic solvents. Such solvates are contemplated as well.

The blood clotting factor compositions is, in various aspects, administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient.

Formulation of the pharmaceutical composition will vary according to the route of administration selected (e.g., solution, emulsion). An appropriate composition comprising the composition to be administered is prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

Pharmaceutical compositions useful in the methods of the present invention containing a blood clotting factor as an active ingredient contain, in various aspects, pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present invention.

A variety of aqueous carriers, e.g., water, buffered water, 0.4% saline, 0.3% glycine, or aqueous suspensions contain, in various aspects, the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

In some embodiments, the blood clotting factor compositions are lyophilized for storage and reconstituted in a suitable carrier prior to use. This technique has been shown to be effective with conventional immunoglobulins. Any suitable lyophilization and reconstitution techniques known in the art are employed. It is appreciated by those skilled in the art that lyophilization and reconstitution leads to varying degrees of antibody activity loss and that use levels may have to be adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above.

In certain embodiments, the concentration of blood clotting factor in these formulations varies widely, for example from less than about 0.5%, usually at or at least about 1% to as much as 15 or 20% by weight and will be selected primarily based on fluid volumes, viscosities, etc., in accordance with the particular mode of administration selected. Thus, for example, and without limitation, a typical pharmaceutical composition for parenteral injection is made up to contain 1 ml sterile buffered water, and 50 mg of blood clotting factor. A typical composition for intravenous infusion could be made up to contain 250 ml of sterile Ringer's solution, and 150 mg of blood clotting factor. Actual methods for preparing parenterally administrable compositions are known or apparent to those skilled in the art and are described in more detail in, for example, Remington's Pharmaceutical Science, 15th ed., Mack Publishing Company, Easton, Pa. (1980). An effective dosage of bispecific antibody is within the range of 0.01 mg to 1000 mg per kg of body weight per administration.

In various aspects, the pharmaceutical compositions are in the form of a sterile injectable aqueous, oleaginous suspension, dispersions or sterile powders for the extemporaneous preparation of sterile injectable solutions or dispersions. The suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In some embodiments, the carrier is a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In all cases the form must be sterile and must be fluid to the extent that easy syringability exists. The proper fluidity is maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms is brought about by various antibacterial and antifungal agents, for example, parabens, chlorobutanol, phenol, sorbic acid, thimerosal, and the like. In many cases, it will be desirable to include isotonic agents, for example, sugars or sodium chloride. In certain aspects, prolonged absorption of the injectable compositions is brought about by the use in the compositions of agents delaying absorption, for example, aluminum monostearate and gelatin.

Compositions useful for administration may be formulated with uptake or absorption enhancers to increase their efficacy. Such enhancers, include, for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

In addition, the properties of hydrophilicity and hydrophobicity of the compositions contemplated for use in the methods or the invention are well balanced, thereby enhancing their utility for both in vitro and especially in vivo uses, while other compositions lacking such balance are of substantially less utility. Specifically, compositions contemplated for use in the invention have an appropriate degree of solubility in aqueous media which permits absorption and bioavailability in the body, while also having a degree of solubility in lipids which permits the compounds to traverse the cell membrane to a putative site of action.

Formulations of recombinant VWF are disclosed in co-owned PCT/US08/88201 and PCT/US08/06291, incorporated herein by reference.

### Preparation of Blood Clotting Factor Polynucleotides and Polypeptides

The elements of the construction of vectors for the expression of a protein product are known to those skilled in the art. Blood clotting factor polynucleotides and polypeptides are expressed in an expression vector comprising a recombinant polynucleotide comprising expression control sequences operatively linked to a nucleotide sequence to be expressed. An expression vector comprises sufficient cis-acting elements for expression; other elements for expression can be supplied by the host cell or in vitro expression system. Expression vectors include all those known in the art, including without limitation cosmids, plasmids (e.g., naked or contained in liposomes) and viruses that incorporate the recombinant polynucleotide. The expression vector is inserted (e.g., via transformation or transduction) into an appropriate host cell for expression of the polynucleotide and polypeptide via transformation or transfection using techniques known in the art. See, e.g., Sambrook, Fritsch & Maniatis, Molecular Cloning: A Laboratory Manual, Second Edition. Cold Spring Harbor, N.Y.: Cold Spring Harbor Laboratory Press, 1989.

Transformation is carried out by a variety of known techniques, depending on the organism, on characteristics of the organism's cells and of its biology. Stable transformation involves DNA entry into cells and into the cell nucleus. For organisms that are regenerated from single cells (which includes some mammals), transformation is carried out in in vitro culture, followed by selection for transformants and regeneration of the transformants. Methods often used for transferring DNA or RNA into cells include micro-injection, particle gun bombardment, forming DNA or RNA complexes with cationic lipids, liposomes or other carrier materials, electroporation, and incorporating transforming DNA or RNA into virus vectors. Other techniques are known in the art. DNA transfer into the cell nucleus occurs by cellular processes, and can sometimes be aided by choice of an appropriate vector, by including integration site sequences which are acted upon by an intracellular transposase or recombinase (see e.g., [Craig, Ann. Rev. Genet. 1988, 22:77; Cox. In Genetic Recombination (R. Kucherlapati and G. R. Smith, eds.) 1988, American Society for Microbiology, Washington, D.C., pages 429-493; Hoess. In Nucleic Acid and Molecular Biology (F. Eckstein and D. M. J. Lilley eds.) Vol. 4, 1990, Springer-Verlag, Berlin, pages 99-109.

Expression vectors and nucleic acids used to express a blood clotting factor in some aspect also contain a tissue-specific promoter. Such promoters are known in the art and include, but are not limited to liver-specific promoters (e.g., albumin; Miyatake et al., J. Virol. 1:5124-32, 1997; α-fetoprotein), muscle-specific promoters (e.g., myosin light chain 1 (Shi et al., Hum Gene Ther. 8:403-10, 1997, α-actin), pancreatic-specific promoter (e.g., insulin or glucagon promoters), neural-specific promoters (e.g., the tyrosine hydroxylase promoter or the neuron-specific enolase promoter), endothelial cell-specific promoters (e.g., von Willebrand factor; Ozaki et al., Hum Gene Ther. 7:1483-90, 1996), and smooth muscle-cells specific promoters (e.g., 22a; Kim et al., J Clin Invest. 100:1006-14, 1997). Other tissue specific promoters include promoters are also being used in developing cancer therapies, including tyrosinase-specific promoters (Diaz et al., J. Virol. 72:789-95, 1998), an adipose tissue promoter derived from human aromatase cytochrome p450 (p450arom) (see U.S. Pat. No. 5,446,143; Mahendroo et al., J. Biol. Chem. 268:19463 19470, 1993; and Simpson et al., Clin. Chem. 39:317 7324, 1993). It is further contemplated that the promoter is an endogenous blood clotting factor promoter. The vectors and other nucleic acid molecules useful in the methods of the invention can also include sequences that limit the temporal expression of the transgene. For example, the transgene is controlled by drug inducible promoters by, for example including cAMP response element enhancers in a promoter and treating the transfected or infected cell with a cAMP modulating drug (Suzuki et al., Hum Gene Ther. 7:1883-93, 1996). Alternatively, repressor elements can prevent transcription in the presence of the drug (Hu et al., Cancer Res 57:3339-43, 1997). Spatial control of expression has also been achieved by using ionizing radiation (radiotherapy) in conjunction with the erg1 gene promoter (Seung et al., Cancer Res 55:5561-5, 1995).

The recombinant nucleic acid constructs encoding human blood clotting factors are inserted into any suitable plasmid, bacteriophage, or viral vector for amplification, and thereby be propagated using methods known in the art, such as those described in Molecular Cloning A Laboratory Manual, 2nd Ed., ed. by Sambrook, Fritsch and Maniatis (Cold Spring Harbor Laboratory Press: 1989). In one embodiment, expression vectors compatible with eukaryotic cells, such as vertebrate cells, are used. Eukaryotic cell expression vectors are well known in the art and are available from commercial sources. Contemplated expression vectors contain both prokaryotic sequences (to facilitate the propagation of the vector in bacteria), and one or more eukaryotic transcription units that are functional in swine cells. Typically, such vectors provide convenient restriction sites for insertion of the desired recombinant DNA molecule. The pcDNAI, pSV2, pSVK, pMSG, pSVL, pPVV-1/PML2d and pIDT1 (ATCC No. 31255) derived vectors are examples of mammalian expression vectors suitable for transfection of non-human cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) are used for expression of proteins in swine cells. The various methods employed in the preparation of the plasmids and the transformation of host cells are well known in the art. For other suitable expression systems for useful in the present invention, as well as general recombinant procedures, see Molecular Cloning A Laboratory Manual, 2nd Ed., ed. by Sambrook, Fritsch and Maniatis (Cold Spring Harbor Laboratory Press: 1989).

Efficient expression from retroviral vectors is observed when “strong” promoters are used to control transcription, such as the SV40 promoter or LTR promoters, reviewed in Chang et al., Int. J. Cell Cloning 7:264, 1989. These promoters are constitutive and do not generally permit tissue-specific expression. Other suitable promoters are discussed herein.

The use of packaging cell lines can increase the efficiency and the infectivity of the produced recombinant virions, see Miller, 1990, Human Gene Therapy 1:5. Murine retroviral vectors have been useful for transferring genes efficiently into murine embryonic, see e.g., Wagner et al., 1985, EMBO J. 4:663; Griedley et al., Trends Genet. 3:162, 1987, and hematopoietic stem cells, see e.g., Lemischka et al., Cell 45:917-927, 1986; Dick et al., Trends in Genetics 2:165-170, 1986.

An additional retroviral technology which permits attainment of much higher viral titers than were previously possible involves amplification by consecutive transfer between ecotropic and amphotropic packaging cell lines, the so-called “ping-pong” method, see e.g., Kozak et al., J. Virol. 64:3500-3508, 1990; Bodine et al., Prog. Clin. Biol. Res. 319: 589-600, 1989. In addition, a techniques for increasing viral titers permit the use of virus-containing supernatants rather than direct incubation with virus-producing cell lines to attain efficient transduction, see e.g., Bodine et al., Prog. Clin. Biol. Res. 319:589-600, 1989. Because replication of cellular DNA is required for integration of retroviral vectors into the host genome, it may be desirable to increase the frequency at which target stem cells which are actively cycling e.g., by inducing target cells to divide by treatment in vitro with growth factors, see e.g., Lemischka et al., Cell 45:917-927, 1986; Bodine et al., Proc. Natl. Acad. Sci. 86:8897-8901, 1989, or to expose the recipient to 5-fluorouracil, see e.g., Mori et al., Jpn. J. Clin. Oncol. 14 Suppl. 1:457-463, 1984.

### Transgenic Animal Preparation

In general, transgenic animals of the invention include any transformable species except humans. Of particular interest are mammals, including known transformable species such as mouse, rat, rabbit, sheep, hamsters, gerbils, guinea pig, and pig, and others, as transformation methods are developed, including bovine and non-human primates.

In one aspect, the transgeneic animals of the invention are genetically modified to prevent expression of multiple endogenous blood clotting factors, particularly Factor VIII and von Willebrand Factor. It is further contemplated that, the transgenic animals of the invention are genetically modified animals in which at least one foreign gene has been inserted into the genome, for example a human VWF is inserted into a FVIII/VWF double knock-out mouse. These animals allow regulatory processes on the cellular level to be examined and influenced in a systematic and specific manner not achievable with other test systems.

Transgenic animals of the type described are useful for analyzing in vivo effects of administration of therapeutic blood clotting factors, including but not limited to Factor VIII (FVIII), von Willebrand Factor (VWF), Factor VII (FVII), Factor IX (FIX), Factor II (FII), Factor V (FV), Factor X (FX), Factor XI, (FXI), Factor XII (FXII), and Factor XIII (FXIII). The transgenic animals serve as excellent models for evaluating the effect of compounds, i.e., purified clotting proteins or variants thereof, on causing the development of anti-self antibodies in context of a putative tolerant host immune system. Such understanding is essential to the design and testing of agents for treatment of blood clotting disorders including, but not limited to, hemophilia, von Willebrand syndrome, and the like.

The transgenes expressing a human polynucleotide sequence as contemplated herein comprise a coding sequence (e.g., cDNA, a synthetic coding sequence, or genomic DNA) for an human blood clotting factor flanked by natural regulatory (expression control) sequences, or associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. It is contemplated that the coding sequence is modified by many means known in the art. Non-limiting examples of such modifications include methylation, “caps,” substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). The control of gene expression is accomplished by a variety of means well-known in the art. Expression of a transgene is constitutive or regulated to be inducible or repressible by known means, typically by choosing a promoter that is responsive to a given set of conditions, e.g., presence of a given compound, or a specified substance, or change in an environmental condition such as tissue type or temperature. The term “inducible expression” extends to any means for causing gene expression to take place under defined conditions, the choice of means and conditions being chosen on the basis of convenience and appropriateness for the host organism.

It is contemplated, in certain aspects, the transgenes are linked to a promoter as described above with respect to recombinant protein expression.

The genetic background of mouse strains from which the various ES cells are derived are known in the art, including ES cells originating from mouse strain 129: R1 cells originate from a mouse blastocyst from a cross between the sub-strains 129/Sv and 129/Sv-CP (Nagy et al., Proc Natl Acad Sci USA. 90:8424-8, 1993); GS1 cells originate from) 29/Sv/Ev. D3-cells (Doetschman et al., Nature 330:576-8, 1987) and J1 cells originate from 129/Sv or 129/terSv. TT2 cells which also yielded ES mice originated from an F1 hybrid strain (C57BL/6×CBA) (Yagi et al., Anal Biochem. 14:70-6, 1993).

Techniques for creating a transgenic animal, particularly a mouse or rat are well known (Gordon, International Review of Cytology 115:171-229, 1989). Various approaches to introducing transgenes are available, including microinjection of nucleic acids into cells, retrovirus vector methods, and gene transfer into embryonic stem (ES) cells. If fertilized oocytes are used for generating a transgenic, desired foreign DNA or transgene is incorporated into the oocytes. Incorporation of the transgene into the oocyte is carried out by several methods such as via an appropriate retroviral vector, or by microinjection. Transgenic mice are generated routinely in the art by microinjection of DNA into blastocysts isolated from pregnant mice, as described in U.S. Pat. No. 4,736,866 issued to Leder et al., and as provided by B. Hogan et al. entitled “Manipulating the Mouse Embryo: A Laboratory Manual”, Cold Spring Harbor Laboratory, Cold Spring Harbor, N.Y., U.S.A. (1986). See also, e.g., Haren et al, Annu. Rev. Microbiol. 53:245-281, 1999; Reznikoff et al., Biochem. Biophys. Res. Commun., 266(3):729-734, 1999; Ivies et al, Methods Cell Bid., 60:99-131, 1999; Hall et al., FEMS Microbiol. Rev. 21:157-178 1997. U.S. Pat. No. 6,492,575 describes a method to of making transgenic mice by transforming ES cells and inject the transformed cells into a tetrapliod blastocyst. By interbreeding heterozygous siblings, homozygous animals carrying the desired gene are obtained.

Additionally, Capecchi et al. describe a method by which transgenes are incorporated into embryonic, fetal or adult pluripotent stem cells (Science 244:1288-1292, 1991). In this method, embryonic stem cells are isolated from blastocysts cultivated in vitro. These embryonic stem cells are kept stable in culture over many cell generations without differentiation. The transgene is then incorporated into the embryonic stem cells by electroporation or other methods of transformation. Stem cells carrying the transgene are selected for and injected into the inner cell mass of blastocysts. The blastocysts are then implanted into pseudopregnant females. Since not all the cells of the inner cell mass of the blastocysts carry the transgenes, the animals are chimeric with respect to the transgenes. Crossbreeding of the chimeric animals allows for the production of animals which carry the transgene. An overview of the process is provided by Capecchi, Trends in Genetics 1989, 5:70-76.

Delivery of the transgene may be accomplished by a retroviral delivery system, see e.g., Eglitis et al., Adv. Exp. Mod Biol. 241:19, 1988. In one aspect, a retroviral construct is one in which the structural genes of the virus are replaced by a single gene which is then transcribed under the control of regulatory elements contained in the viral long terminal repeat (LTR). A variety of single-gene-vector backbones have been used, including the Moloney murine leukemia virus (MoMuLV). In one embodiment, retroviral vectors which permit multiple insertions of different genes such as a gene for a selectable marker and a second gene of interest, under the control of an internal promoter are derived from this type of backbone, see e.g., Gilboa, Adv. Exp. Med. Biol. 241:29, 1988.

It is contemplated, in some embodiments, that the during the introduction of the transgene into the animal, the transgene is inserted into the endogenous gene, thereby knocking-out function of the endogenous gene. In other embodiments, the exogenous gene is inserted into the animal genome in a location such that the expression of the endogenous gene is preserved. Thus, the transgenic animal may express all or part of the endogenous polynucleotide that corresponds to the human transgene polynucleotide inserted into the animal.

### Dosing of Exogenous Blood Factors

The dosage regimen involved in a method for treating a condition described herein with a human blood clotting factor will be determined by the attending physician, considering various factors which modify the action of drugs, e.g. the age, condition, body weight, sex and diet of the patient, the severity of any infection, time of administration and other clinical factors. By way of example, a dose of a recombinant VWF useful in the present invention is approximately 50 U/kg, equal to 500 μg/kg, approximately 75 U/kg, approximately 100 U/kg, approximately 150 U/kg, approximately 200 U/kg, approximately 250 U/kg, approximately 300 U/kg, approximately 350 U/kg, approximately 400 U/kg, approximately 450 U/kg or approximately 500 U/kg, or the equivalent μg/kg or mg/kg.

In certain embodiments, formulations of the invention are administered by an initial bolus followed by a continuous infusion to maintain therapeutic circulating levels of drug product. As another example, the inventive compound is administered as a one-time dose. Those of ordinary skill in the art readily optimize effective dosages and administration regimens as determined by good medical practice and the clinical condition of the individual patient. The frequency of dosing depend on the pharmacokinetic parameters of the agents and the route of administration. The pharmaceutical formulation is determined by one skilled in the art depending upon the route of administration and desired dosage. See for example, Remington's Pharmaceutical Sciences, 18th Ed. (1990, Mack Publishing Co., Easton, Pa. 18042) pages 1435-1712, the disclosure of which is hereby incorporated by reference. Such formulations may influence the physical state, stability, rate of in vivo release, and rate of in vivo clearance of the administered agents. Depending on the route of administration, a suitable dose may be calculated according to body weight, body surface area or organ size. Appropriate dosages may be ascertained through use of established assays for determining blood level dosages in conjunction with appropriate dose-response data. The final dosage regimen will be determined by the attending physician, considering various factors which modify the action of drugs, e.g. the drug's specific activity, the severity of the damage and the responsiveness of the patient, the age, condition, body weight, sex and diet of the patient, the severity of any infection, time of administration and other clinical factors. As studies are conducted, further information will emerge regarding the appropriate dosage levels and duration of treatment for various diseases and conditions.

### Kits

As an additional aspect, the invention includes kits which comprise one or more pharmaceutical formulations for administration of exogenous blood factor to a patient packaged in a manner which facilitates their use for administration to subjects. In one embodiment, such a kit includes pharmaceutical formulation described herein (e.g., a composition comprising a therapeutic protein or peptide), packaged in a container such as a sealed bottle or vessel, with a label affixed to the container or included in the package that describes use of the compound or composition in practicing the method. In one embodiment, the pharmaceutical formulation is packaged in the container such that the amount of headspace in the container (e.g., the amount of air between the liquid formulation and the top of the container) is very small. Preferably, the amount of headspace is negligible (i.e., almost none). In one embodiment, the kit contains a first container having a therapeutic protein or peptide composition and a second container having a physiologically acceptable reconstitution solution for the composition. In one aspect, the pharmaceutical formulation is packaged in a unit dosage form. The kit may further include a device suitable for administering the pharmaceutical formulation according to a specific route of administration. Preferably, the kit contains a label that describes use of the pharmaceutical formulations.

The following examples are not intended to be non-limiting example, exemplary of specific embodiments of the invention.

### EXAMPLES

### Example 1

### Effect of Exogenous VWF on FVIIIxVWD Double Knockout Mice

Mouse models of human hemophilia A and von Willebrand disease (VWD) have been used for pharmacokinetic, pharmacodynoamic and immunological studies of FVIII or VWF. FVIII deficient mice lack FVIII protein but carry normal levels of endogenous mouse VWF, which might interact differently with infused human FVIII than human VWF. Therefore a mouse model with FVIII deficiency and with human VWF instead of murine VWF would be advantageous for the study of hemophilia. In order to study the effects of human VWF in hemophilia, a transgenic mouse line was established based on mice deficient in both endogenous FVIII and VWF (FVIIIxVWD).

Intravenous injection of human VWF was not an effective option for bringing the VWF-FVIII co-deficient mice back to normal circulating levels of VWF over time due to the relatively short half-life of human VWF in vivo in these animals (approximately 5 hours). As such, an alternative method of delivery of human VWF was developed.

High molecular weight human VWF was injected intraperitoneally (i.p.) or subcutaneously (s.c.) into FVIIIxVWD-mice to determine if these routes were effective in recovering circulating plasma levels of VWF in deficient animals. Mice were treated with a recombinant VWF (rVWF) purified from CHO cells co-expressing rFVIII and VWF Turecek et al., Hamostaseologie, 2009 (submitted); Plaimauer et al., Semin Thromb Hemost. 27:395-403, 2001; Parti et al., Häemophilia 11:492-6, 2005; Shapiro et al. Vasc Health Risk Manag. 3:555-65, 2007). The starting material for the rVWF product was an intermediate of the manufacturing process of the commercial rFVIII product ADVATE® as described in Blanchette V S, Shapïro A D, Liesner R J, Hernández Navarro F, Warner I, Schroth P C, Spotts G, Ewenstein B M; rAHF-PFM Clinical Study Group. Plasma and albumin-free recombinant factor VIII: pharmacokinetics, efficacy and safety in previously treated pediatric patients. J Thromb Haemost. 2008; 6:1319-26.

Mice were injected with human VWF i.p. with 400 IU VWF:Ag/kg body weight (10 ml/kg) and given a repeat injection 9 hours after the first injection. Formulation buffer was used as control. Blood was collected by heart punctures from treated and control animals (n=5) at 2, 6, 9, 11 and 23 after the first i.p. injection. VWF plasma levels were determined by enzyme linked immunosorbant assay (ELISA) using two polyclonal anti-VWF antibodies (Dakopatts, Glostrup, Denmark) and developed using ortho-phenylenediamine color development (Sigma, St, Louis, Mo.).

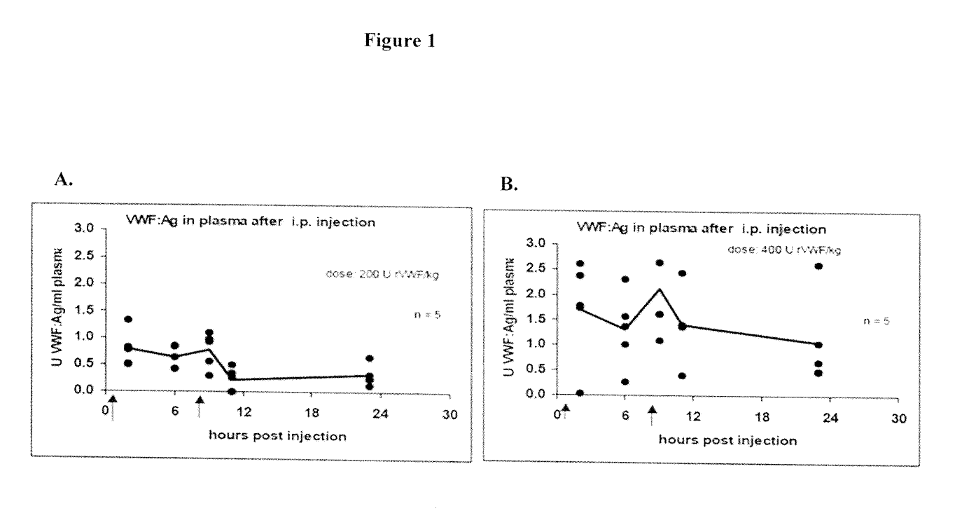

VWF antigen levels found in mouse plasma 2 hours after i.p. injection showed a mean level of 1.7±0.9 VWF:Ag units/ml (FIG. 1B), indicating up lasma recovery of 17% of i.p. injected VWF. The second injection of rVWF (at 9 hours) increased the mean VWF:Ag to 2.1 VWF:Ag unit/ml. At 23 hours post injection there were still measurable amounts of circulating VWF:Ag of approximately 1.1 VWF:Ag units/ml. The study was repeated at a lower dose of 200 U/kg with the same treatment intervals of 2 hours and 9 hours. The level of plasma VWF:Ag observed at the lower dose was approximately 3-fold less than seen at the higher dose, representing approximately 12% of recovery in the circulation (FIG. 1A).

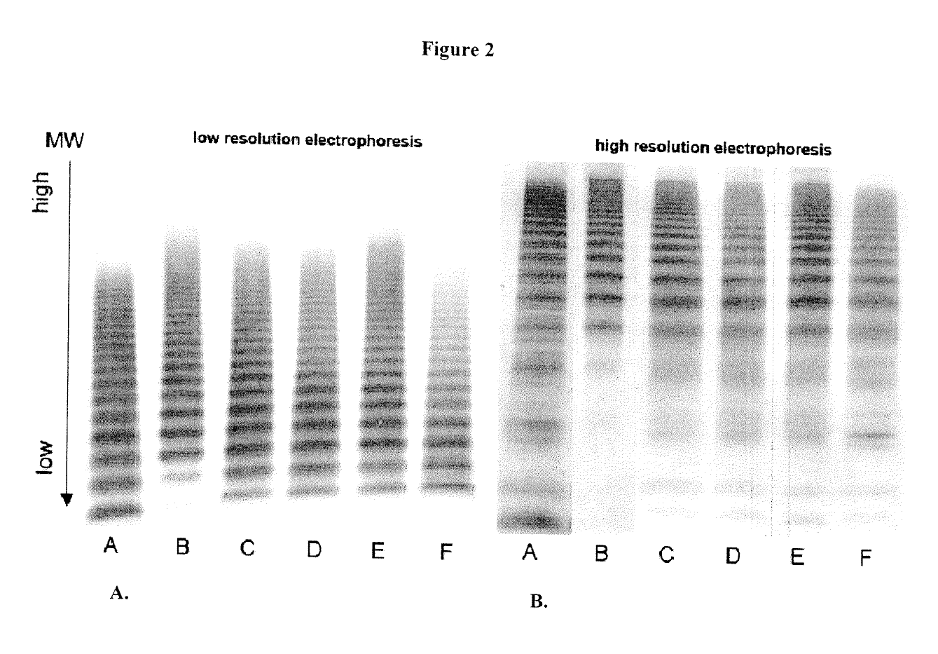

Surprisingly, the VWF found in the circulation showed an intact multimer pattern that was comparable to the multimer pattern of the injected VWF material. The rVWF that was used for treatment of mice showed a lower content of dimer and corresponding degradation bands, but still had a multimer pattern with high molecular weight multimers that started to be reduced and degraded after 9 hours (FIG. 2).

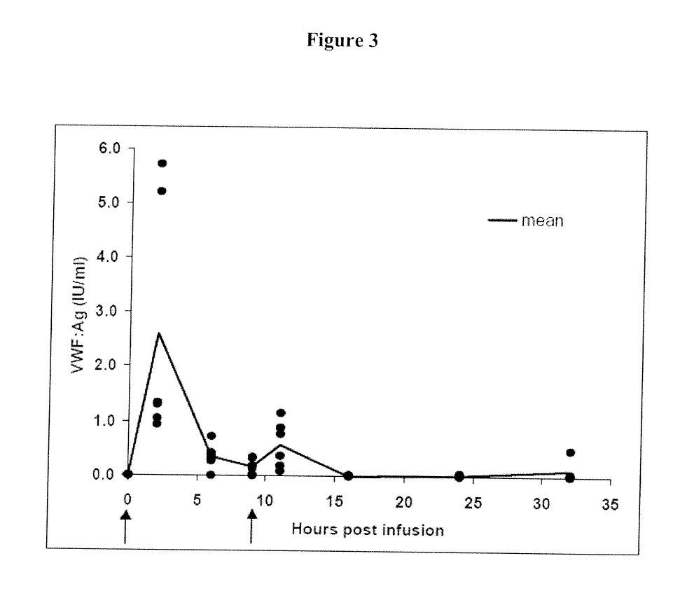

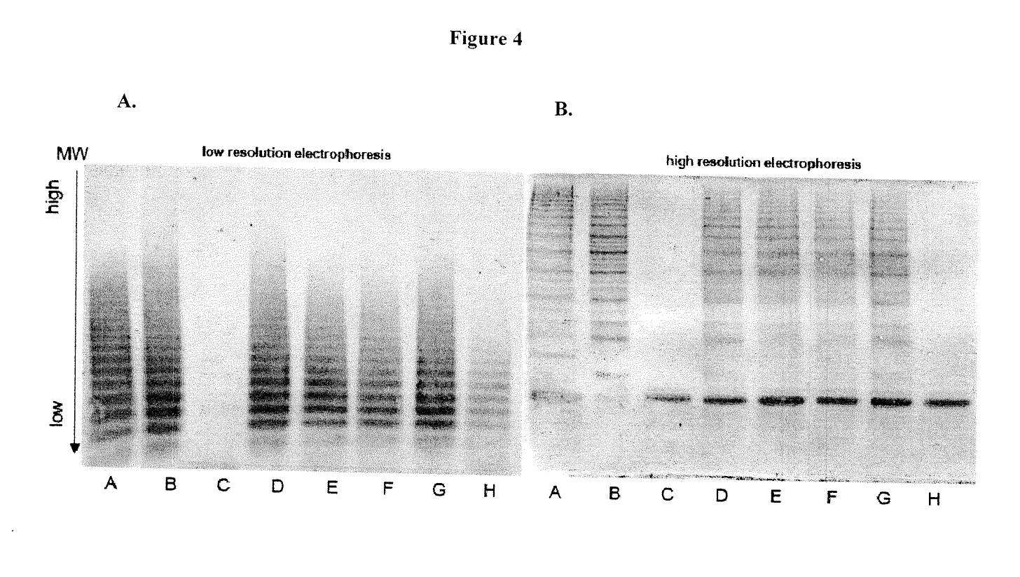

Experiments were also carried out to determine if plasma derived VWF (pdVWF), often used to treat hemophilia patients, was also detectable as circulating multimers. Mice were treated with a similar treatment as above with two i.p. injections of pdVWF and the levels of VWF were measured as above. As observed with recombinant VWF, injection of pdVWF resulted in circulating VWF antigen levels and intact multimers with a multimer pattern similar to that of the injected protein (FIGS. 3 and 4).

These results demonstrate that both recombinant or plasma derived human VWF is detectable in the circulation of FVIIIxVWD-mice as early as 2 hours after i.p injection, and as late as 23 hours post injection. Thus, i.p injection of VWF demonstrates a longer half life in vivo than intravenous injection of the same solution, making i.p injection a better treatment option for patients undergoing VWF therapy.

### Example 2

### Administration of FVIII/VWF Complex to FVIIIxVWD Knockout Mice

The finding that intact VWF multimers were observed after i.p. injection of rVWF or pdVWF into mice lacking VWF led to the investigation of whether a complex of human rFVIII and rVWF could also be administered by i.p injection with a similar outcome.

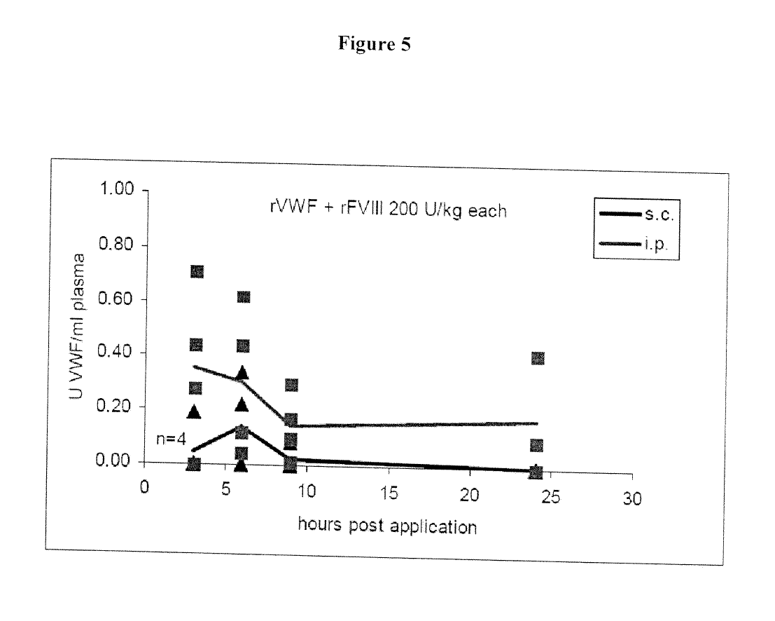

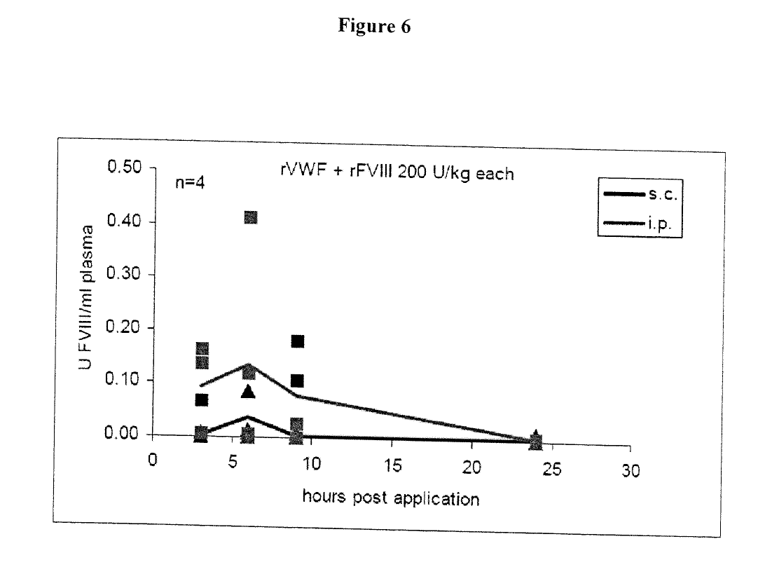

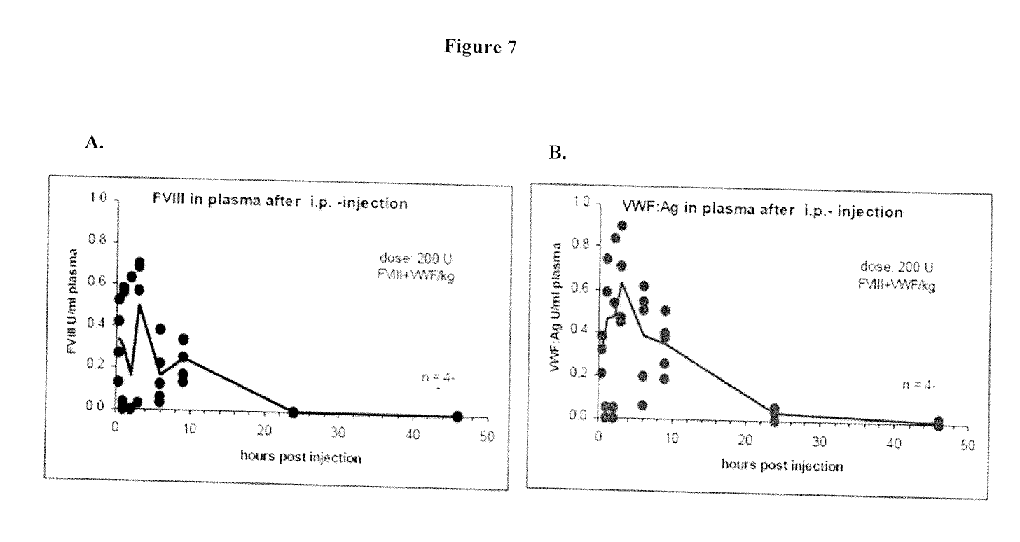

Combinations of each of 200 IU rFVIII/kg and 200 IU VWF:Ag/kg were injected i.p. in the same treatment intervals as described above. Analysis of blood as above demonstrated that circulating VWF:Ag levels were detectable for up to 24 hours, similar to animals treated with VWF alone (FIG. 5). FVIII levels measured at the same timepoints showed that FVIII was detectable at 0.1±0.6 units/ml after 3 hours and maintained this level up to 9 hours. FVIII was not detectable after 24 hours (FIG. 6). For these experiments, recovery of VWF after i.p. injection was approximately 12% of injected material while FVIII recovery was approximately 5%. In a repeat experiment, blood levels were determined for up to 48 hours. The FVIII and VWF levels seen up to 24 hours were similar to the previous experiments, but FVIII and VWF were not detectable in the circulation after 46 hours (FIG. 7).

FVIIIxVWD double knockouts were also administered rVWF or FVIII via subcutaneous injection of a complex containing 200 IU rFVIII/kg and 200 IU VWF:Ag/kg. After s.c. injection, there was a small increase in circulating VWF:Ag levels (0.14 VWF:Ag units/ml) (FIG. 1). Corresponding FVIII levels were low, but detectable (0.04 units/ml) at 6 hours post injection (FIG. 6).

These results demonstrate that i.p. injection is a successful route of administration of the large VWF molecule, demonstrating a better recovery in circulation than intravenous or s.c. injection. Although i.p. injection is widely used for administration of small molecule drugs in pharmacological studies in non-human species (e.g., i.p injection of erythropoietin), the i.p. injection of proteins, particularly of the large size of VWF multimers, has not been investigated until now. Due to the large molecular weight of multimeric VWF it was unexpected that this protein would be recovered intact in the circulation of mice after i.p. injection. This indicates that the large multimeric protein be transported through the mesothelioma into the vasculature while maintaining the structure and function of the large VWF protein. This newly discovered ability of rVWF to transport into the vasculature provides a novel approach to the study of hemophilia A with circulating human VWF.

### Example 3

### Assessment of Exogenous Human VWF on Blood Clotting in FVIIIxVWD Knockouts

Similar to human hemophilia, FVIIIxVWD double knockout mice present with a tendency to have bleeding episodes. Administration of exogenous human VWF to FVIIIxVWD double knockout mice demonstrated that intraperitoneal, and to a lesser extent subcutaneous, injection of rVWF or pdVWF allows for VWF to be transported into the vasculature and demonstrated detectable activity in the circulation. This observation indicates that the exogenous VWF likely exhibits characteristic VWF activity in vivo, including attracting platelets to the site of vascular injury, mediating platelet-platelet interaction, and stabilizing Factor VIII (FVIII) in the circulation (Plaimauer et al., Semin Thromb Hemost. 27:395-403, 2001).

Collagen is a physiological binding partner of VWF. Studies by Kessler et al. (Blood 63:1291-1298, 1984) have shown that the complex of VWF and Factor VIII binds to collagen fibrillae. U.S. Pat. No. 6,414,125 teaches that only high or medium molecular weight VWF binds to collagen, and provides methods for detecting VWF binding to collagen and purification of VWF using collagen immobilization. As such, VWF-collagen binding assays are also a measure of the presence of high molecular weight VWF species after i.p. or s.c. administration of VWF in vivo.

To determine the effects of exogenous VWF on blood clotting and other related VWF activities, in vivo assays to measure platelet adhesion, platelet aggregation, or blood clotting/blood flow are carried out using techniques known in the art.

For example, U.S. Pat. No. 6,005,077 discloses methods for determining the effects of blood factor deficiency on bleeding times and blood characteristics (e.g. hemoglobin content, presence of coagulation factors) before and after administration of exogenous blood clotting factor such as VWF. Additionally, platelet adhesion assays are disclosed in Shenkman et al. (Thromb Haemost. 96:160-6, 2006) which describes a method for measuring platelet adhesion under flow conditions using immobilized recombinant VWF fragments. de Romeuf et al. (Thromb Haemost. 79:211-6, 1998) discloses in vitro platelet activation and platelet aggregation assays using a preparation of rVWF.

These assays are useful to assess the ability of exogenous blood clotting factor to effect blood clotting disorders and provide information regarding dosing and administration of exogenous factor to ameliorate disease.

In addition, assays to characterize VWF and to diagnose VWD are performed as described by others. For example, methods to determine the role of VWF in hemorrhagic and thrombotic disorders is described in Franchini, et al., Crit. Rev Clin Lab Sci. 44(2):115-49 (2007); methods to assess treatment of VWD and VWF therapy are described in Budde et al., Semin Thromb Hemost. September; 32(6):626-35 (2006), Favaloro Semin Thromb Hemost. September;32(6):566-76 (2006) and Favaloro, Semin Thromb Hemost. 2006 July; 32(5):456-71 (2006); methods for classification of VWD are described in Sadler et al., J Thromb Haemost. October;4(10):2103-14 (2006); and methods for identification of VWD are described in Kouides Semin Thromb Hemost. July; 32(5):480-4 (2006).

### Example 4

### Treatment of Blood Clotting Disorders Using Exogenous Human Blood Clotting Factor

Subjects having a deficiency in a blood clotting factor, such as FVIII and VWF are treated with blood factor compositions as described herein. Administration of blood factor(s) in animal models of blood clotting disorders and using protocols known in the art to treat humans suffering from blood disorders provides the basis for administering subjects the blood factor(s) described herein alone or in combination with other therapeutic agents, e.g., chemotherapeutic or radiotherapeutic agents, cytokines, growth factors, and other commonly used therapeutics.

For example, hemophilia A patients having a deficiency in FVIII are treated with low-PEGylated FVIII at therapeutically effective doses, as is readily determined by the treating physician. See for example, Di Paola et al., Haemophilia. 13:124-30, 2007, which describes administration and comparison of two different preparations of replacement FVIII to patients with severe hemophilia.

Purified VWF has been used to treat patients suffering von Willebrands disease (Majumdar et al., Blood Coagul Fibrinolysis 4:1035-7, 1993) as well as hemophilia A. Recombinant or plasma-derived VWF as described herein is used in regimens known to those of skill in the art to treat patients who would benefit from replacement VWF.

It is contemplated in the present invention that recombinant VWF is administered alone or in a complex with FVIII. In one embodiment, the VWF is administered in a dose range from 50 U/kg to 500 U/kg. For example, VWF is administered at approximately 50, 100, 150, 200, 250, 300, 350, 400, 450, or 500 U/kg.

Administration of the blood factors may last 1-24 hours, or longer, and is amenable to optimization using routine experimentation. It is also contemplated that the blood factor is given for a duration not requiring extended treatment. Additionally, modified blood factor composition are administered daily, weekly, bi-weekly, or at other effective frequencies, as would be determinable by one of ordinary skill in the art.

It is contemplated that a modified blood factor is administered to patients in combination with other therapeutics, such as with other chemotherapeutic or radiotherapeutic agents, or with growth factors or cytokines. When given in combination with another agent, the amount of modified blood factor is reduced accordingly if necessary. Second agents are administered in an amount determined to be safe and effective at ameliorating human disease.

In certain embodiments, cytokines or growth factors, and chemotherapeutic agents or radiotherapeutic agents are administered in the same formulation as blood factor and given simultaneously. Alternatively, the agents are administered in a separate formulation and are still administered concurrently with blood factor. As used herein, concurrently refers to agents given within 30 minutes of each other. In a related embodiment, the second agent is administered prior to administration of modified blood factor. Prior administration refers to administration of the agent within the range of one week prior to modified blood factor treatment up to 30 minutes before administration of modified blood factor. It is further contemplated that the second agent is administered subsequent to administration of modified blood factor. Subsequent administration is meant to describe administration from 30 minutes after modified blood factor treatment up to one week after modified blood factor administration. Blood factor compositions are administered in conjunction with a regimen of radiation therapy in a subject having a blood clotting disorder and a form of cancer, treatment being carried out as prescribed by a treating physician.

Numerous modifications and variations in the invention as set forth in the above illustrative examples are expected to occur to those skilled in the art. Consequently only such limitations as appear in the appended claims should be placed on the invention.

1. A transgenic non-human animal having a genome lacking a functional, endogenous Factor VIII (FVIII) gene and lacking a functional, endogenous von Willebrand Factor (VWF) gene.

2. The transgenic animal of claim 1, further having a genome comprising a human transgene polynucleotide sequence encoding human VWF.

3. The transgenic animal of claim 2, wherein the polynucleotide sequence is operably linked to a promoter polynucleotide sequence.

4. The transgenic animal of claim 2, wherein the polynucleotide sequence comprises a polyadenylation polynucleotide sequence.

5. The transgenic animal of claim 1, wherein the animal is a rodent.

6. The transgenic animal of claim 5, wherein the animal is a mouse.

7. A non-human transgenic animal having a genome lacking a functional, endogenous Factor VIII (FVIII) gene and lacking a functional, endogenous von Willebrand Factor (VWF) gene, said genome comprising a polynucleotide encoding a human von Willebrand Factor, said human VWF having physiological activity of the human VWF, said transgenic mammal having in its genome an exogenous transgene construct comprising:
(a) transcriptional regulatory polynucleotide sequences,
(b) DNA encoding said human VWF, and
(c) a polyadenylation signal,
wherein (A), (B) and (C) are operably linked in said exogenous gene construct to obtain production of said human VWF or fragment thereof in said transgenic animal.

8. The transgenic animal of claim 7, wherein the transcriptional regulatory polynucleotide sequences are selected from the group consisting of 5′ transcriptional regulatory polynucleotide sequences, 3′ transcriptional regulatory polynucleotide sequences, internal transcriptional regulatory polynucleotide sequences, and combinations thereof.

9. The transgenic animal of claim 8, wherein the 5′ regulatory sequence is a promoter, optionally comprising an enhancer region.

10. The transgenic animal of claim 1 or 7, wherein the animal is an experimental model of human acquired hemophilia A.

11. A method for assessing the effects of exogenous human von Willebrand factor (VWF) on a mammal deficient in endogenous Factor VIII and endogenous VWF comprising administering to the animal of any one of claims 1 to 10 a therapeutically effective amount of human VWF and measuring a clinical readout of VWF activity, wherein an improvement in clinical readout indicates a therapeutic effect of exogenous VWF.

12. The method of claim 11 wherein the VWF is administered intraperitoneally or subcutaneously.

13. The method of claim 12 wherein the VWF is administered in a range from 50 VWF:Ag U/kg to 500 U/kg.

14. The method of claim 11 wherein the clinical readout of VWF activity is selected from the group consisting of blood composition, platelet aggregation, platelet adhesion and platelet activation.

15. A method for treating a subject having hemophilia A comprising administering an exogenous multimeric human von Willebrand Factor in an amount effective to ameliorate a symptom of hemophilia A, wherein the VWF is administered intraperitoneally or subcutaneously.

16. The method of claim 15 wherein the symptom is selected from the group consisting of bleeding disorder, autoimmune disease, aberrant platelet aggregation, aberrant platelet adhesion and aberrant platelet activation.

17. The method of claim 15 wherein the VWF is administered in a range from 50 VWF:Ag U/kg to 500 U/kg.

18. The method of claim 11 or 15 wherein the VWF is administered in combination with human Factor VIII.
